(19) United States
(12) Patent Application Publication
(10) Pub. No.: US 2021/0260080 A1
(43) Pub. Date: Aug. 26, 2021

(54) Prenatal Supplement Formulation and Method of Administration

(71) Applicant: Sharai Amaya, Greenwood, SC (US)

(72) Inventor: Sharai Amaya, Greenwood, SC (US)

(21) Appl. No.: 16/870,050

(22) Filed: May 8, 2020

Related U.S. Application Data
(60) Provisional application No. 62/979,260, filed on Feb. 20, 2020.

(51) Int. Cl. A61K 31/616; A61K 31/07; A61K 31/355; A61K 31/593; A61K 31/375; A61K 31/122; A61K 31/51; A61K 31/455; A61K 31/675; A61K 31/714; A61K 31/4188; A61K 31/519; A61K 31/525; A61K 33/06; A61K 33/26; A61K 33/18; A61K 33/30; A61K 33/34; A61K 33/32; A61K 33/22; A61K 33/04; A61K 33/00; A61K 31/14; A23L 33/15; A23L 33/155; A23L 33/16; A23L 33/115; A23L 33/00

(52) U.S. Cl. CPC A61K 31/616; A23V 2002/00; A61K 31/355; A61K 31/593; A61K 31/375; A61K 31/122; A61K 31/51; A61K 31/455; A61K 31/675; A61K 31/714; A61K 31/4188; A61K 31/519; A61K 31/525; A61K 33/06; A61K 33/26; A61K 33/18; A61K 33/30; A61K 33/34; A61K 33/32; A61K 33/22; A61K 33/04; A61K 33/00; A61K 31/14; A23L 33/15; A23L 33/155; A23L 33/16; A23L 33/115; A23L 33/40; A61K 31/07

(57) ABSTRACT

A prenatal supplement formulation for treating and preventing preeclampsia and other prenatal conditions and method of using the same. The prenatal supplement formulation is provided in a single unit dosage form. The prenatal supplement formulation comprises a therapeutically effective dosage of aspirin and a dietary supplement compound. The dietary supplement compound comprises a multi-vitamin component, a mineral component, and a nutritional component. A method of treating or preventing a prenatal disease, condition, or disorder using the prenatal supplement formulation is also provided.

### CROSS-REFERENCE

This application claims priority from Provisional Patent Application Ser. No. 62/979,260 filed on Feb. 20, 2020.

### BACKGROUND

Many expectant mothers are unfortunately exposed to risks for preeclampsia, pulmonary embolism, clotting disorders, and other adverse thrombotic events. Other pregnancy related risks that can occur include low birth weight babies, preterm birth, stillbirth, and other adverse pregnancy outcomes. Certain expectant mothers that are at higher risk for these conditions require additional medical intervention. Aspirin is not recommended in low-risk pregnancies. However, low-dose aspirin (i.e. acetylsalicylic acid) is recommended for higher risk mothers for these additional potential complications.

Maternal risk factors for preeclampsia include a personal history of preeclampsia, multifetal gestation, chronic hypertension, type 1 or 2 diabetes, renal disease, autoimmune disease, nulliparity (no prior births), obesity, mother ow sister with preeclampsia, low socioeconomic status, African American race or other minority, age greater than 35 years, personal history of low birth weight infant, previous adverse pregnancy outcome, and greater than 10-year interpregnancy interval. This broad scope of risk factors affects large portions of the obstetric population.

Most expectant mothers take a prenatal vitamin to supplement their diet with the essential nutrients required for a healthy pregnancy. Adding supplemental low-dose aspirin to the prenatal vitamin is a logical addition for numerous high risk expectant mothers. However, without a convenient combination of a prenatal vitamin supplement that also contains the recommended dosage of low-dose aspirin, expectant mothers may not take aspirin at all or may not take the correct dose of aspirin. With an increasing number of medications required, expectant mothers may be less likely to adhere to prescribed treatments and may forget to take both the prenatal vitamin supplement and the aspirin. Without a well formulated prenatal vitamin supplement, expectant mothers may have nutritional gaps in their diet and may not be getting all of the essential vitamins, minerals, and nutrients needed during pregnancy and the postpartum period.

Accordingly, there is a great need for a unique pharmaceutical composition suitable for higher risk pregnancies. The present invention combines the benefits of low-dose aspirin with a prenatal vitamin supplement to help ensure that a pregnant woman consumes the daily recommended amount of aspirin to prevent preeclampsia and other dangerous conditions, and improves convenience and compliance with therapy for pregnant women by offering a combined supplement that may be taken daily during a pregnancy. The pharmaceutical composition further reduces the number of medications and vitamin supplements that must be taken each day by combining the two products.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a prenatal supplement formulation. The prenatal supplement formulation is provided in a single unit dosage form. The single unit dosage form may be a pill, a tablet, a capsule, a chewable, a dissolving powder, or a liquid. The prenatal supplement formulation comprises a therapeutically effective dosage of aspirin. The therapeutically effective dosage of aspirin is typically a low-dose aspirin dosage for treating or preventing a prenatal disease, condition, or disorder.

The prenatal supplement formulation further comprises a dietary supplement compound. The dietary supplement compound comprises a multi-vitamin component, a mineral component, and a nutritional component. The multi-vitamin component comprises vitamin A, vitamin C, vitamin D3, vitamin E, vitamin K2, thiamin, riboflavin, niacin, vitamin B5, vitamin B6, vitamin B12, biotin, and folate. The mineral component comprises calcium, iron, iodine, magnesium, zinc, copper, choline, chromium, manganese, molybdenum, selenium, and boron. The nutritional component may comprise docosahexaenoic acid and eicosapentaenoic acid, or a blend of Omega-3 fatty acids and at least one probiotic.

An additional embodiment of the present invention comprises a method of treating or preventing a prenatal disease, condition, or disorder in a pregnant human. The method comprises orally administering a prenatal supplement formulation in a single unit dosage form to an expectant mother. The prenatal supplement formulation comprises a therapeutically effective dosage of aspirin. The therapeutically effective dosage of aspirin is typically a low-dose aspirin dosage for treating or preventing a prenatal disease, condition, or disorder.

The prenatal supplement formulation further comprises a dietary supplement compound. The dietary supplement compound comprises a multi-vitamin component, a mineral component, and a nutritional component. The multi-vitamin component comprises vitamin A, vitamin C, vitamin D3, vitamin E, vitamin K, thiamin, riboflavin, niacin, vitamin B5, vitamin B6, vitamin B12, biotin, and folate. The mineral component comprises calcium, iron, iodine, magnesium, zinc, copper, choline, chromium, manganese, molybdenum, selenium, and boron. The nutritional component may comprise docosahexaenoic acid and eicosapentaenoic acid, or a blend of Omega-3 fatty acids and at least one probiotic. The prenatal supplement formulation is administered once per day.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention discloses a unique combination of a prenatal vitamin and a recommended dose of aspirin for use by pregnant women at risk for certain adverse pregnancy outcomes. The combination drug is available in various forms, such as tablets, chewable tablets, capsules, soft gel capsules, gummies, liquids, dissolving powders, and the like. This invention ensures that an appropriate dose of aspirin is combined with other prenatal supplement ingredients to help prevent preeclampsia, thromboembolic events, low birth weight, stillbirth, preterm birth and other related pregnancy conditions while ensuring the proper nutrition of a fetus and mother during pregnancy. Differing embodiments of the combination can be delivered in different dosage delivery mediums. Exact specifications may vary during manufacturing.

The term “therapeutically effective dose” as used herein is meant to refer to an amount of a composition or compound effective to result in the amelioration of symptoms associated with a condition, or to provide a beneficial therapeutic effect, such as, but not limited to, as treating or preventing a prenatal disease, condition, or disorder, such as, preeclampsia, pulmonary embolism, clotting disorders, other adverse thrombotic events, low birth weight babies, preterm birth, and other adverse pregnancy outcomes.

The term “single unit dosage form” as used herein is meant to refer to the co-administration of at least two compounds within the same time frame, such as substantially simultaneously. The at least two compounds are combined in a single delivery form, such as, but not limited to, a pill, a capsule, a tablet, a chewable, a gummy, a liquid, a dissolving powder, and the like.

One embodiment of the present invention comprises a prenatal supplement formulation for treating or preventing a prenatal disease, condition, or disorder. Common prenatal diseases, conditions, or disorders include preeclampsia, pulmonary embolism, clotting disorders, thrombotic events, preterm birth, low weight babies, stillbirth, and other adverse pregnancy outcomes. The prenatal supplement formulation may be administered once per day or more or less as prescribed.

The prenatal supplement formulation is provided in a single unit dosage form. The single unit dosage form may be a pill, a tablet, a capsule, a chewable, a gummy, a powder, a liquid, or any other form known to one of skill in the art. This is desirable as it allow for the administration of different therapeutic components in a single delivery form so that the patient is more likely to comply with a recommended or prescribed dosage regimen. The prenatal supplement formulation may comprise an enteric coating or similar polymer barrier to ensure the release of the therapeutic components in the small intestine instead of the stomach.

The prenatal supplement formulation comprises a therapeutically effective dose of aspirin (acetylsalicylic acid). The therapeutically effective dose of aspirin is generally a range of about 70-650 mg with about 81-150 mg being a typical dose, although it may be more or less as the patient's condition indicates. The prenatal supplement formulation further comprises a dietary supplement compound. The dietary supplement compound comprises a multi-vitamin component. The multi-vitamin component is typically a mixture of vitamins common in multi-vitamins and prenatal vitamins. In one example, the multi-vitamin component comprises vitamin A, vitamin C as calcium ascorbate, vitamin D3 as vegan cholecalciferol, vitamin E, vitamin K2 as natural menaquinone-7, vitamin B1(thiamin), vitamin B2 (riboflavin), vitamin B3 (niacin), vitamin B6 as pyridoxine HCL, vitamin B12 as methylcobalamin, biotin, and folate as 5MTHF. However, this is not meant as a limitation, as the multi-vitamin component may include any combination of these vitamins, or any other known vitamins or sources of vitamins as well.

The dietary supplement compound further comprises a mineral component. The mineral component is typically a mixture of minerals common in multi-vitamin supplements and prenatal vitamin supplements. In one example, the mineral component comprises calcium from mineralized seaweed and calcium ascorbate, iron as ferronyl carbonyl iron (advantageous as an easily digestible and absorbable form), iodine from kelp (advantageous as a vegan form), magnesium as magnesium oxide, magnesium asparate, and magnesium glycinate, zinc as zinc cholate and zinc picolinate, copper, choline, chromium as chromium amino acid chelate, manganese, molybdenum, selenium, and boron as calcium fructoborate. However, this is not meant as a limitation, as the mineral component may include any combination of these minerals, or any other known minerals or sources of minerals as well.

The dietary supplement compound further comprises a nutritional component. The nutritional component is typically a mixture of nutrients common in multi-vitamin supplements and prenatal vitamin supplements. In one example, the nutritional component comprises docosahexaenoic acid and eicosapentaenoic acid. The nutritional component may further comprise a blend of Omega-3 fatty acids and at least one probiotic, such as, but not limited to Lactobacillus, Bifidobacterium, or the like. The nutritional component may further comprise a combination of probiotics, whole food fruit, and vegetable blend. However, this is not meant as a limitation, as the nutritional component may include any combination of these nutrients, or any other known nutrients or sources of nutrients as well. For example, whole foods are advantageous as they provide phytonutrients that are not yet fully understood and not reproducible in individual forms.

The pharmaceutical formulation may further contain one or more binders with adhesive properties that serve to promote cohesiveness in the solid dosage form. The formulation may contain one or more disintegrants to promote acceptable disintegration rate in the gastrointestinal tract. “Disintegrant” means a material in the formulation that acts to cause the solid matrix to break up when the solid dosage form comes in contact with aqueous media. The formulation may contain lubricants to prevent pharmaceutical ingredients from clumping together during manufacturing. The formulation may contain excipients, meaning materials other than the two active pharmaceutical ingredients (acetaminophen and NSAID), such as glints, flavorants, preservatives, and dilutants. Excipient ingredients may be used in making a film coating.

The following are nonlimiting examples provided to further illustrate formulations of the present invention.

### EXAMPLES

### Formulation 1

A tablet of the prenatal supplement formulation is prepared with the following ingredients and dosages:

| Ingredient | dosage | |
|---|---|---|
| Acetylsalicylic Acid (Aspirin) | 81 | mg |
| Vitamin A | 770 | IU |
| Vitamin C | 100 | mg |
| Vitamin D3 | 25 | mcg |
| Vitamin E | 15 | mg |
| Vitamin K | 90 | mcg |
| Vitamin B1 | 1.5 | mg |
| Vitamin B2 | 1.4 | mg |
| Vitamin B3 | 18 | mg |
| Vitamin B6 | 2 | mg |
| Vitamin B12 | 200 | mcg |
| Biotin | 30 | mcg |
| Folate | 1000 | mcg |
| Calcium | 100 | mg |
| Iron | 18 | mg |
| Iodine | 150 | mcg |
| Magnesium | 400 | mg |
| Zinc | 10 | mg |
| Copper | 1000 | mcg |
| Choline | 450 | mcg |
| Chromium | 30 | mcg |
| Manganese | 2 | mg |
| Molybdenum | 50 | mcg |
| Selenium | 60 | mcg |
| Boron | 1 | mg |
| Docosahexaenoic Acid | 175 | mg |
| Eicosapentaenoic Acid | 88 | mg |

This formulation represents one embodiment of a typical dosage of the prenatal supplement formulation for the treatment of prenatal diseases, conditions, or disorders include preeclampsia, pulmonary embolism, clotting disorders, thrombotic events, preterm birth, low weight babies, and other adverse pregnancy outcomes. The dosages of ingredients may vary depending on the specific requirements or needs of the patient. The formulation may also include an additional blend of Omega-3 fatty acids, probiotics, and a whole food fruit and vegetable blend.

### Formulation 2

A tablet of the prenatal supplement formulation is prepared with the following ingredients and dosages:

| Ingredient | dosage range | |
|---|---|---|
| Acetylsalicylic Acid (Aspirin) | 150 | mg |
| Doxylamine | 20 | mg |
| Vitamin A | 750 | IU |
| Vitamin C | 120 | mg |
| Vitamin D (cholecalciferol) | 20 | mcg |
| Vitamin E | 13.5 | mg |
| Vitamin K | 45 | mcg |
| Vitamin B1 (thiamine) | 1.4 | mg |
| Vitamin B2 (riboflavin) | 1.4 | mg |
| Vitamin B3 (niacin) | 7 | mg |
| Vitamin B6 (pyridoxine) | 25 | mg |
| Vitamin B12 | 2.6 | mcg |
| Biotin | 30 | mcg |
| Folate | 800 | mcg |
| Calcium | 250 | mg |
| Iron | 6 | mg |
| Iodine | 220 | mcg |
| Magnesium | 360 | mg |
| Zinc | 7.5 | mg |
| Copper | 9000 | mcg |
| Choline | 300 | mcg |
| Chromium | 50 | mcg |
| Manganese | 5 | mg |
| Molybdenum | 50 | mcg |
| Selenium | 50 | mcg |
| Boron | 1 | mg |
| Taurine | 50 | mg |
| Pantothenic Acid (Vitamin B5) | 6 | mg |
| Vanadium | 10 | mcg |
| Docosahexaenoic Acid | 200 | mg |
| Eicosapentaenoic Acid | 15 | mg |

This formulation represents one embodiment of a typical dosage of the prenatal supplement formulation for the treatment of prenatal diseases, conditions, or disorders. Advantages of this formulation include the addition of doxylamine for the prevention of pregnancy related nausea and vomiting, Vitamin B6 at higher doses (25 mg) has been shown to effectively reduce nausea in pregnancy, higher doses of magnesium may help to prevent constipation by promoting gastrointestinal motility, Vitamin B5 (pantothenic acid), and vanadium for the control of blood sugar.

### Formulation 3

A single unit dosage of the prenatal supplement formulation is prepared with the following ingredients and ranges of dosages:

| Ingredient | dosage range | |
|---|---|---|
| Acetylsalicylic Acid (Aspirin) | 40-650 | mg |
| Doxylamine | 0-50 | mg |
| Vitamin A | 0-6000 | IU |
| Vitamin C | 50-2000 | mg |
| Vitamin D (cholecalciferol) | 5 mcg-3,000 mg | |
| Vitamin E | 7.5-20 | mg |
| Vitamin K | 0-100 | mcg |
| Vitamin B1 (thiamine) | 0.75-3 | mg |
| Vitamin B2 (riboflavin) | 0.425-3.4 | mg |
| Vitamin B3 (niacin) | 0-34 | mg |
| Vitamin B6 (pyridoxine) | 1-50 | mg |
| Vitamin B12 | 1-500 | mcg |
| Biotin | 0-100 | mcg |
| Folate | 400-4000 | mcg |
| Calcium | 0-5,000 | mg |
| Iron | 0-130 | mg |
| Iodine | 50-300 | mcg |
| Magnesium | 0-800 | mg |
| Zinc | 2-30 | mg |
| Copper | 0-10,000 | mcg |
| Choline | 0-3,500 | mcg |
| Chromium | 0-240 | mcg |
| Manganese | 0-10 | mg |
| Molybdenum | 0-109 | mcg |
| Selenium | 0-400 | mcg |
| Boron | 0-20 | mg |
| Taurine | 0-5,000 | mg |
| Pantothenic Acid (Vitamin B5) | 0-12 | mg |
| Vanadium | 0-50 | mcg |
| Docosahexaenoic Acid | 0-2000 | mg |
| Eicosapentaenoic Acid | 0-1500 | mg |

This formulation represents a typical dosage range of ingredients of the prenatal supplement formulation.

An additional embodiment of the present invention comprises a method for treating or preventing a prenatal disease, condition, or disorder in a pregnant human. The method comprises orally administering a prenatal supplement formulation to the pregnant human once per day. The prenatal supplement formulation is administered in a single unit dosage form as discussed supra. The single unit dosage form may be a pill, a tablet, a capsule, a chewable, a gummy, a powder, a liquid, or any other form known to one of skill in the art.

The prenatal supplement formulation comprises a therapeutically effective dose of aspirin, typically about 81-150 mg, although it may be more or less as the patient's condition indicates. The prenatal supplement formulation further comprises a dietary supplement compound. The dietary supplement compound comprises a multi-vitamin component. The multi-vitamin component is typically a mixture of vitamins common in multi-vitamins and prenatal vitamins as discussed supra.

The dietary supplement compound further comprises a mineral component. The mineral component is typically a mixture of minerals common in multi-vitamin supplements and prenatal vitamin supplements as discussed supra. The dietary supplement compound further comprises a nutritional component. The nutritional component is typically a mixture of nutrients common in multi-vitamin supplements and prenatal vitamin supplements as discussed supra.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A prenatal supplement formulation in a single unit dosage form comprising:
a therapeutically effective dosage of aspirin; and
a dietary supplement compound.

2. The prenatal supplement formulation of claim 1, wherein the therapeutically effective dosage of aspirin is about 81 mg.

3. The prenatal supplement formulation of claim 1, wherein the single unit dosage form is a pill, a tablet, a capsule, a chewable, a dissolving powder, or a liquid.

4. The prenatal supplement formulation of claim 1 further comprising an enteric coating.

5. The prenatal supplement formulation of claim 1, wherein the prenatal supplement composition is administered once per day.

6. The prenatal supplement formulation of claim 1, wherein the dietary supplement compound comprises a multi-vitamin component.

7. The prenatal supplement formulation of claim 6, wherein the multi-vitamin component comprises vitamin A, vitamin C, vitamin D3, vitamin E, vitamin K2, thiamin, riboflavin, niacin, vitamin B6, vitamin B12, biotin, and folate.

8. The prenatal supplement formulation of claim 1, wherein the dietary supplement compound comprises a mineral component.

9. The prenatal supplement formulation of claim 8, wherein the mineral component comprises calcium, iron, iodine, magnesium, zinc, copper, choline, chromium, manganese, molybdenum, selenium, and boron.

10. The prenatal supplement formulation of claim 1, wherein the dietary supplement compound comprises a nutritional component.

11. The prenatal supplement formulation of claim 10, wherein the nutritional component comprises docosahexaenoic acid and eicosapentaenoic acid.

12. The prenatal supplement formulation of claim 1, wherein the dietary supplement compound comprises a multi-vitamin component, a mineral component, and a nutritional component.

13. A prenatal supplement formulation in a single unit dosage form for treating or preventing a prenatal disease, condition, or disorder, the prenatal supplement formulation comprising:
a therapeutically effective dosage of aspirin; and
a dietary supplement compound comprising a multi-vitamin component, a mineral component, and a nutritional component.

14. The prenatal supplement formulation of claim 13, wherein the therapeutically effective dosage of aspirin is about 81 mg to 150 mg.

15. The prenatal supplement formulation of claim 13, wherein the multi-vitamin component comprises vitamin A, vitamin C, vitamin D3, vitamin E, vitamin K2, thiamin, riboflavin, niacin, vitamin B6, vitamin B12, biotin, and folate.

16. The prenatal supplement formulation of claim 13, wherein the mineral component comprises calcium, iron, iodine, magnesium, zinc, copper, choline, chromium, manganese, molybdenum, selenium, and boron.

17. The prenatal supplement formulation of claim 13, wherein the nutritional component comprises docosahexaenoic acid and eicosapentaenoic acid.

18. The prenatal supplement formulation of claim 13, wherein the nutritional component comprises a blend of Omega-3 fatty acids and at least one probiotic.

19. A method of treating or preventing a prenatal disease, condition, or disorder in a pregnant human, comprising:
orally administering a prenatal supplement formulation in a single unit dosage form comprising:
a therapeutically effective dosage of aspirin; and
a dietary supplement compound comprising a multi-vitamin component, a mineral component, and a nutritional component; and
wherein the prenatal supplement formulation is administered once per day.

20. The method of claim 19, wherein the therapeutically effective dosage of aspirin is about 81 mg to 150 mg.
